(19) United States
(12) Patent Application Publication
(10) Pub. No.: US 2005/0129759 A1
(43) Pub. Date: Jun. 16, 2005

(54) Sustained release compositions and controlled delivery method

(76) Inventor: Milan Sojka, Algonquin, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/736,968

(22) Filed: Dec. 16, 2003

(51) Int. Cl. A61K 9/52; A61K 9/26

(52) U.S. Cl. 424/469

(57) ABSTRACT

A sustained release composition comprising expanded thermoplastic microspheres, an active agent, and an optional release retardant is disclosed. The composition has an improved ability to stabilize and release the active agent over an extended time period.

### FIELD OF THE INVENTION

The present invention relates to the controlled and sustained release of an active agent from a composition over an extended time. More particularly, the present invention relates to a composition that exhibits a sustained and controlled release of an active agent, wherein the composition comprises expanded thermoplastic microspheres, an active agent incorporated therein, and an optional release retardant coating over the active agent and expanded thermoplastic microspheres.

### BACKGROUND OF THE INVENTION

The use of porous, absorbent, and/or intersticed materials as a means for retaining active agents is known. The active agents range widely in properties for use in a wide range of applications, including therapeutic, cosmetic, food, pharmaceutical, hygienic, and industrial applications. Examples of products embodying such materials include personal care products, cosmetics, toiletries, fragrances, pesticides, catalysts, polymerization initiators, photoinitiators, pharmaceutical products, household products, and industrial products.

An important feature of these products is an ability to extend their utility over a long period of time by retaining a sizeable quantity of the active agent, while slowly releasing the active agent over time to perform its intended function. Control of the release rate for the active agent is achieved in a variety of ways, including diffusion from a porous or absorbent material, forcing the active agents to the product surface by compression, and rupture of internal bubbles or cells in a matrix material.

The controlled release of an active agent, such as a drug or cosmetic compound, improves the safety, efficacy, and reliability of a treatment regimen that utilizes the active agent. Conventional dosage forms for delivering an active agent often provide a wide variation in the amount of active agent that is available during treatment. Consequently, the treatment regimen requires multiple doses such that the concentration of active agent is maintained at its minimum effective level. In particular, conventional dosage forms quickly release the active agent, which causes a sharp increase in active agent concentration to a peak, followed by a sharp decline in active agent concentration. This wide swing in active agent concentration often provides initial acceptable results, but inadequate treatment as active agent concentrations decrease over time.

This problem can be overcome by administering an effective dose of an active agent in a conventional delivery system at more frequent intervals. However, individuals find such treatment regimens inconvenient, which leads to eliminating or delaying treatment doses, thereby adversely affecting the efficacy of the treatment.

In contrast, controlled release of an active agent regulates the release rate of the active agent and reduces the frequency of treatment doses, thereby improving compliance with the treatment regimen. Ideally, a controlled release of an active agent provides a predictable amount of the active agent for effective treatment, and controls the rate of active agent release over a predetermined time. The controlled release of an active agent can occur at a constant rate, at a constant declining rate, or at some other specified rate or pattern to achieve an efficacious release of the active agent.

The controlled release of an active agent has several advantages including fewer compliance problems during the treatment regimen, utilizing less of the active agent during treatment, improving efficacy of the treatment, and an overall cost savings. Although such benefits are recognized in the art, it has been difficult to provide compositions that achieve a sustained and controlled release of an active agent.

It has been especially difficult to achieve a controlled release of a water-soluble active agent when the water-soluble agent is a component of an aqueous formulation, or when the water-soluble agent, in its controlled release form, is subjected to an aqueous medium. In these situations, the water-soluble agent has a tendency to be released too quickly.

Conversely, it is difficult to achieve a controlled release of an oil-soluble active agent when the oil-soluble active agent is a component of an oil-based formulation or when the oil-soluble active agent, in its controlled release form, is subjected to a nonaqueous medium. In this situation, the oil-soluble agent has a tendency to be released too quickly.

For example, a water-soluble or an oil-soluble active agent can be converted into a controlled release form by adsorbing the active agent onto an adsorbent polymer. The resulting controlled release form of the active compound can be formulated into a solid composition, e.g., a tablet or powder, a semisolid composition, e.g., a cream or gel, or a liquid composition, e.g., an emulsion or dispersion. Prior compositions have demonstrated a premature release of a water-soluble active agent when the controlled release form of the active agent is incorporated into an aqueous medium, like an emulsion, or when a semisolid or solid composition contacts an aqueous medium. Similarly, there is a premature release of an oil-soluble active agent when the controlled release form of the active agent is incorporated into a nonaqueous medium, like a body oil, or when the composition contacts a non-aqueous medium.

In addition, many active agents have inherent stability problems, such as a tendency to oxidize over time, a tendency to degrade in the presence of moisture and/or light, or a sensitivity to shock. It would be an improvement in the art if such inherently unstable active ingredients can be formulated into a composition that overcomes these stability problems, and provides a sustained and controlled release of the active agent over an extended time.

U.S. Pat. No. 5,593,680 discloses cosmetic and dermopharmaceutical compositions having expanded thermoplastic microspheres dispersed in an aqueous gel. The microspheres are utilized to improve the physical properties of the gel and to improve the ease of application and gentleness of the composition. The compositions have no disclosed sustained release properties, and the active ingredient is not incorporated into the microspheres for sustained release.

### SUMMARY OF THE INVENTION

The present invention is directed to compositions that provide a sustained and controlled release of an active agent, including water-soluble and oil-soluble active agents. The controlled release compositions can be used as is, or incorporated into formulations, including liquids, gels, semisolids, and solids. More particularly, the present invention is directed to a controlled release composition comprising expanded thermoplastic microspheres and an active agent. The active agent is incorporated into the expanded thermoplastic microspheres. In certain embodiments, the expanded thermoplastic microspheres treated with the active agent are coated with a release retardant to further assist a sustained and controlled release of the active agent.

Therefore, one aspect of the present invention is to provide a controlled release composition comprising expanded thermoplastic microspheres, an active agent that is incorporated into the expanded thermoplastic microspheres, and, an optional release retardant coated on the active agent-treated expanded thermoplastic microspheres. The controlled release compositions impart stability to inherently unstable active agents and an improved controlled release of the active agent.

Another aspect of the present invention is to provide a controlled release composition comprising (a) expanded thermoplastic microspheres, having incorporated therein (b) a water-soluble or an oil-soluble active agent in an amount up to twenty times the weight of the expanded thermoplastic microspheres, and (c) an optional release retardant that coats and/or is adsorbed onto the expanded thermoplastic microspheres and active agent. The active agent can be a solid or a liquid compound at room temperature.

Yet another aspect of the present invention is to provide a controlled release composition comprising a water-soluble active agent, and incorporating the controlled release composition into an aqueous or nonaqueous formulation that exhibits a controlled release and delivery of the water-soluble agent.

Another aspect of the present invention is to provide a controlled release composition comprising an oil-soluble active agent, and incorporating the controlled release composition into an aqueous or nonaqueous formulation, like an oil, that exhibits controlled release and delivery of the oil-soluble agent.

Still another aspect of the present invention is to provide a controlled release composition containing an active ingredient selected from the group consisting of a skin care compound, a hair care compound, a topical drug, an antioxidant, a dye, a self-tanning compound, a skin-lightening compound, an optical brightener, a deodorant, a fragrance, a sunscreen, a pesticide, a drug, a catalyst, a polymerization initiator, a photoinitiator, a pigment, a metal or metal salt, and similar compounds, and mixtures thereof, incorporated into expanded thermoplastic microspheres.

These and other aspects and novel features of the present invention will become apparent from the following detailed description of the preferred embodiments.

### DETAILED DESCRIPTION OF THE PREFERRED EMBODIMENTS

The present sustained release compositions function as controlled delivery systems for an active agent incorporated into expanded thermoplastic microspheres. In particular, release of an active agent from the expanded thermoplastic microspheres occurs in a sustained manner, providing a continuous supply of the active agent to the tissues, medium, or area that the sustained release composition contacts.

The present invention provides advantages over prior sustained release compositions. For example, one advantage of a present sustained release composition is the ability of the composition to release a low, efficacious amount of the active agent over a broad area and an extended time in a substantially uniform manner, thereby avoiding waste and further controlling the activity level of the active agent.

The amount of active agent retained in the expanded thermoplastic microspheres awaiting release is held in reserve with minimal exposure to the atmosphere. For those active agents that are volatile and produce irritating vapors, retention in the expanded thermoplastic microspheres reduces the rate of volatilization. At the same time, the amounts of active agent held in reserve are excluded from contact with the contacted surface until their release, thus lessening any high initial effect and preventing undesirable side effects to contacted surfaces.

The present specification is directed primarily to compositions containing topically active agents. For example, the sustained release composition can be used topically on skin or hair. However, the active agent can be a different type of compound, such as a fragrance, which is control released to act as a room deodorizer, or a pesticide, which is released in a controlled manner for extended insecticidal or herbicidal activity, or similar types of active agents, like drugs and therapeutic agents, that are used in sustained and controlled release applications. Other sustained release compositions of the present invention can be designed for application to inanimate surfaces. The sustained release composition also can have industrial applications, for example, the controlled release of a catalyst or polymerization initiator. The sustained release composition of the present invention further can be used in the food industry for the controlled and sustained release of spices, flavors, and food colors.

Therefore, with respect to some active agents, it is desirable to topically deliver the active agents to human skin or hair. In many cases, the active agents can be applied directly to the skin, either in a substantially pure form or in a liquid vehicle. A direct application, however, is limited in a number of respects. First, direct application allows rapid evaporation of volatile active agents. Second, application of an active agent in a substantially pure form can cause toxic and/or allergic reactions, particularly in the case of infrared absorbents, insect repellents, and steroids. Finally, many topically applied active agents have undesirable esthetic properties, such as an oily feel or a strong odor. While such disadvantages often can be minimized by dilution of the active agent in a suitable liquid carrier, the decrease in active agent concentration can limit the effectiveness of the resulting product for its intended purpose.

Because of such disadvantages, it is desirable to provide compositions capable of providing a sustained and controlled delivery of an active agent after it has been applied to the skin or hair. Desirably, such controlled delivery compositions also control odor or toxicity associated with the active agent and should be suitable both for direct application to the skin and for application in combination with conventional carriers.

The present invention provides sustained release compositions incorporating a variety of active agents, such as ultraviolet absorbants (sunscreens), optical brighteners, insect repellants, steroids, acne treatments, epidermal lipid replacements, counterirritants, hair growth promoters, and the like. The sustained release compositions can be used as is, or can be incorporated into a carrier or vehicle, alone or with other formulation ingredients.

A sustained release composition of the present invention is a dry, free-flowing product that can be rubbed directly on the skin, for example, to provide a controlled release of the active agent over time. More typically, the sustained release composition is formulated with a carrier vehicle and other ingredients. The use of a formulation containing a sustained release composition avoids incompatibilities, chemical or physical, that might otherwise exist between the active agent and a second active ingredient in the formulation, or between the active substance and the carrier or other formulation ingredients. The sustained release compositions also protect inherently unstable active agents from oxidation, chemical degradation, exposure to light, and other physical and chemical instabilities, including premature evaporation.

The controlled release of the active agent achieved by a sustained release composition of the present invention provides a prolonged activity of the active agent, e.g., on the skin, for example. This prolonged activity reduces the need to frequently reapply the active agent. Additionally, controlled release of the active agent reduces any odor associated with the active agent and reduces the possibility of a toxic or allergic reaction resulting from direct contact of the active agent with the skin.

A controlled release composition of the present invention comprises: (a) expanded thermoplastic microspheres, (b) an active agent, and (c) an optional release retardant. The active agent is incorporated into the expanded thermoplastic microspheres. The optional release retardant is coated on the expanded thermoplastic microspheres containing the active agent.

As used herein, the term “incorporated” is defined as one or more of adsorbing, absorbing, coating, or impregnating an active agent into or onto the expanded thermoplastic microspheres.

A present controlled release composition is a dry, free-flowing powdery material, and can be formulated into (a) a solid product in the form of a powder, or tablet, for example, (b) a semisolid, like a cream or gel, for example, or (c) a liquid, like an emulsion or dispersion, for example.

More particularly, the present controlled release compositions comprise expanded thermoplastic microspheres having an active agent incorporated therein. The weight amount of active agent applied to the expanded thermoplastic microspheres can be up to about twenty times the weight of the expanded thermoplastic microspheres. Typically, the amount of active agent applied to the expanded thermoplastic microspheres is equal to and up to about fifteen times the weight of the expanded thermoplastic microspheres. Preferably, the amount of active agent applied to the expended thermoplastic microspheres is equal to and up to about ten times the weight of the expanded thermoplastic microspheres. The active agent can be water soluble or oil soluble, and can be a solid or a liquid.

An optional release retardant is applied to the expanded thermoplastic microsphere-active agent combination to adsorb onto the expanded thermoplastic microspheres and/or coat the expanded thermoplastic microspheres and active agent. The release retardant can be water soluble or dispersible, i.e., is hydrophobic, or oil soluble or dispersible, i.e., is hydrophobic. If the active agent is water soluble, the release retardant preferably is hydrophobic. If the active agent is oil soluble, the release retardant preferably is hydrophilic.

As used herein, the term “water-soluble compound” is defined as a compound having a solubility in water of at least 0.5 g per 100 grams of water at 25° C. Similarly, “oil-soluble compound” is defined as a compound having a solubility in mineral oil of at least 0.5 g per 100 grams of mineral oil at 25° C. The terms “water dispersible” and “oil dispersible” are defined as compounds having a solubility, at 25° C., in 100 g of water or mineral oil, respectively, of about 0.1 to about 0.5 g.

A sustained release composition of the present invention can be used as is for a time-extended delivery of the active agent. Similarly, the composition can be formulated with other ingredients to provide a powder or tablet, a semisolid, or a liquid formulation for a time extended delivery of the active agent. For example, a present sustained release composition can be applied topically, such that the active ingredient, e.g., an antioxidant, is slowly released from the expanded thermoplastic microspheres, over an extended time, to perform its intended function.

Surprisingly, the present sustained release compositions are sufficiently stable, even in liquid formulations, such that the compositions retain their controlled release properties until administered, e.g., applied to the skin. Previously, liquid formulations containing a sustained release composition, like those containing a water-soluble active ingredient, exhibited poor controlled release properties because water present in a formulation displaced the active agent from the its absorbent substrate, thereby solubilizing or dispersing the active agent. A similar disadvantage is observed with respect to sustained release compositions containing an oil-soluble active agent, and formulation of the sustained release composition into a nonaqueous solvent. In either case, the active agent becomes available for immediate use, but is not available for a controlled release from the absorbent substrate after application of the sustained release composition.

The individual components of the present sustained release compositions are discussed in more detail below.

1. Expanded Thermoplastic Microspheres

Expanded thermoplastic microspheres are known, and can be prepared, for example, as set forth in European Patent Application 0112807 and U.S. Pat. Nos. 3,615,972; 3,864,181; 4,397,799; 4,513,106; and 4,722,943, each incorporated herein by reference.

The expanded thermoplastic microspheres can be produced from any nontoxic and nonirritant thermoplastic material. Homopolymers and copolymers of acrylonitrile or vinylidene chloride can be used as the material of construction for the expanded thermoplastic microspheres, for example. A typical, but nonlimiting, expanded thermoplastic microsphere is a copolymer containing, by weight, 0% to about 60% monomer units derived from vinylidene chloride, about 20% to about 90% monomer units derived from acrylonitrile, and 0% to about 50% monomer units derived from an acrylic or styrene monomer, the sum of the weight percentages being 100%. The acrylic monomer can be, for example, a methyl or ethyl acrylate or methacrylate. The styrene monomer can be, for example, α-methylstyrene or styrene. Additional monomers useful in the preparation of the expanded thermoplastic microspheres are disclosed in U.S. Pat. Nos. 3,615,972 and 3,864,181, each incorporated herein by reference. The expanded thermoplastic microspheres can be in a dry state or hydrated state.

The internal cavity of the expanded thermoplastic microspheres contains a gas, which can be a hydrocarbon, such as isobutene or isopentane, or, alternatively, air. Useful expanded thermoplastic microspheres include those marketed under the brand name EXPANCEL® available from Akzo Nobel, Sundsvail, Sweden, especially microparticles of the DE (dry expanded state) grade.

Especially useful expanded thermoplastic microspheres are EXPANCEL® DE GRADES 551 DE 40 d42, 551 DE 20 d60, 551 DE 80 d42, 461 DE 40 d60, 461 DE 20 d70, 051 DE 40 d60, 091 DE 40 d30, 091 DE 80 d30, and mixtures thereof. The expanded thermoplastic microspheres typically have a median particle size of about 5 to about 250, and preferably about 10 to about 100, microns (μm), and particle size range of about 0.1 to about 1000 μm, and preferably about 1 to about 500 μm. The expanded thermoplastic microspheres have a density of about 20 to about 200, and preferably about 25 to about 80, kg/m³.

The weight amount of expanded thermoplastic microspheres in a present sustained release composition is about 5% to about 99.9%, and preferably about 8% to about 95%, by weight, of the total composition. To achieve the full advantage of the present invention, the amount of expanded thermoplastic microspheres in a sustained release composition is about 10% to about 80%, by weight, of the total composition. The particular weight amount of expanded thermoplastic microspheres present in a sustained release composition of the present invention is related to the identity and amount of active agent in the composition. The amount of a particular active agent required to perform its intended function first is determined, then the amount of expanded thermoplastic microspheres is determined based on considerations such as the identity of the expanded thermoplastic microspheres and active agent, and the ability of the active agent to adsorb, absorb, coat, and impregnate the expanded thermoplastic microspheres. Such a determination is easily performed by persons skilled in the art.

2. Active Agent

In accordance with an important feature of the present invention, the active agent can be any of a wide variety of compounds, either water soluble or oil soluble. The active agent can be a liquid or a solid compound at room temperature (25° C.). Often, the active agent is a topically active compound. The sustained release composition, therefore, can be applied to the skin, and the active agent then performs its intended function as it is released from the sustained release composition over time and contacts the skin.

The active agent incorporated into the expanded thermoplastic microspheres can be in liquid or solid form. The active agent can be a liquid at room temperature that is incorporated into the expanded thermoplastic microspheres neat or dissolved in a suitable solvent. The active agent also can be a solid at room temperature that is dissolved, dispersed, or suspended in a suitable carrier for incorporation into the expanded thermoplastic microspheres. Dispersed or suspended particles of solid active agent have a particle size that is smaller than the pore size of the expanded thermoplastic microspheres, typically less than about one micron.

After application, a liquid or solid active agent diffuses out of the expanded thermoplastic microspheres upon rubbing contact or contact with surfaces or media with which the sustained release composition is placed in contact. An active agent in solid form can be delivered to the contacted area by gradually dissolving into the bodily secretions at the points of exposure or into a surrounding liquid medium, for example.

The active agent often is a water-soluble or water-dispersible compound, i.e., is hydrophilic. However, the active agent can be oil soluble or oil dispersible, i.e., is hydrophobic. In other embodiments, the active agent is a mixture of compounds, either all hydrophilic, all oleophilic, or a mixture of hydrophilic and oleophilic compounds. As discussed hereafter, the optional release retardant also may contribute to the efficacy of the composition.

The active agent is present in the sustained release composition in an amount sufficient to perform its intended function, typically in an amount of about 0.1% to about 95%, by weight, of the composition, and preferably about 5% to about 85%, by weight, of the composition. To achieve the full advantage of the present invention, the active agent is present in an amount of about 20% to about 50%, by weight, of the sustained release composition.

A present sustained release composition can be incorporated into liquid, solid, or semisolid formulations that contain the required, or desired, amount of active agent. Persons skilled in the art are aware of the amount of active agent needed to perform its intended function, and are capable of determining the amount active agent to incorporate into a sustained release composition based on the form, e.g., solid, semisolid, or liquid, of the formulation. Alternatively, a sustained release composition of the present invention can be used as prepared, i.e., is not incorporated into a formulation.

Any liquid or soluble solid active agent, either polar or nonpolar, can be incorporated into the expanded thermoplastic microspheres. Water-soluble active agents, like dyes, ascorbic acid, hyularonic acid, hydrogen peroxide, salts of active agents, proteins, and enzymes, or water-insoluble active agents, like retinol, tocopherol, hydroquinone, kojic acid, salicylic acid, alpha and beta hydroxyacids, metals, catalysts, polymerization initiators, photoinitiators, oxidants, peroxides, pigments, reductants, polymers, adsorbents, fragrances, and vitamins, can be used.

With respect to topically active agents, such agents are intended to be applied to the skin or hair, and allowed to remain on the skin or hair for an extended time period to allow a controlled release of the active agent to perform its function.

The topically active agent, therefore, can be one of, or a mixture of, a cosmetic compound, a medicinally active compound, or any other compound that is useful upon topical application to the skin or hair. Such topically active agents include, but are not limited to, hair-growth promoters, deodorants, skin-care compounds, plant extracts, antioxidants, insect repellents, counterirritants, vitamins, steroids, retinoids, hair dyes, antibacterial compounds, antifungal compounds, antiinflammatory compounds, topical anesthetics, sunscreens, optical brighteners, and other cosmetic and medicinal topically effective compounds.

For example, a skin or hair conditioner can be the active agent of a composition of the present invention. Skin conditioning agents include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, almond oil, grape seed oil, and castor oil, and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin or hair conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to:
myristyl propionate,
isopropyl isostearate,
isopropyl myristate,
isopropyl palmitate,
cetyl acetate,
cetyl propionate,
cetyl stearate,
isodecyl neopentanoate,
cetyl octanoate,
isocetyl stearate;

(b) aliphatic di- and tri-esters of polycarboxylic acid, including, but not limited to:
diisopropyl adipate,
diisostearyl fumarate,
dioctyl adipate, and
triisostearyl citrate;

(c) aliphatic polyhydric alcohol esters, including, but not limited to:
propylene glycol dipelargonate;

(d) aliphatic esters of aromatic acids, including, but not limited to:
C₁₂-C₁₅ alcohol esters of benzoic acid,
octyl salicylate,
sucrose benzoate, and
dioctyl phthalate.

Numerous other esters are listed in the CTFA Handbook, at pages 24 through 26, incorporated herein by reference.

In addition, the topically active agent can be a hair dye, such as, but not limited to, m-aminophenol hydrochloride, p-aminophenol sulfate, 2,3-diaminophenol hydrochloride, 1,5-naphthalene-diol, p-phenylenediamine hydrochloride, sodium picramate, cationic dyes, anionic dyes, FD&C dyes, like Blue No. 1, Blue No. 2, Red No. 3, Red No. 4, or Red No. 40, D&C dyes, like Yellow No. 10, Red No. 22, or Red No. 28, and pyrogallol. Numerous other hair dyes are listed in the CTFA Handbook, pages 70-71, incorporated herein by reference.

The active agent also can be a hydrocarbon, like mineral oil, 1-decene dimer, a polydecene, paraffin, petrolatum, or an isoparaffin, for example.

An especially useful class of active agents is the silicone oils, like dimethicone, and the functional silicone oils, like dimethicone copolyol. The silicone oils have a viscosity of about 10 centipoise (cps) to about 600,000 cps, and typically about 350 cps to about 10,000 cps, at 25° C. Examples of silicone oils include dimethicone, dimethicone copolyol, dimethiconol, simethicone, phenyl trimethicone, stearoxy dimethicone, trimethylsilylamodinethicone, an alkyl dimethicone copolyol, and a dimethicone having polyoxyethylene and/or polyoxypropylene side chains.

The topically active agent also can be an antioxidant, like ascorbic acid or erythorbic acid, or an optical brightener, like a distyrylbiphenyl derivative, stilbene or a stilbene derivative, a pyralozine derivative, or a coumarin derivative. In addition, a self-tanning compound, like dihydroxy acetone, or a hair growth promoter can be the topically active agent.

Optical brighteners useful as the active agent can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine.

One class of optical brighteners is the bistriazinyl derivatives of 4,4′-diaminostilbene-2,2′-disulfonic acid, exemplified in Table 1.

TABLE 1

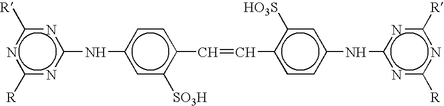

| R | R′ |
|---|---|
| —NHC₆H₅ | —OCH₃ |
| —NHC₆H₅ | —NHCH₃ |
| —NHC₆H₅ | 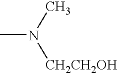 |
| —NHC₆H₅ | —N(CH₂CH₂OH)₂ |
| —NHC₆H₅ | 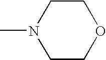 |
| —NHC₆H₅ | —NHC₆H₅ |
| 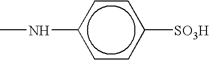 | —N(CH₂CH₂OH)₂ |
| 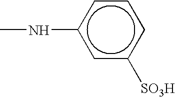 | —N(CH₂CH₂OH)₂ |
| 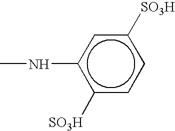 | —N(CH₂CH₃)₂ |

Additional classes of optical brighteners are the 2-(stilben-4-yl)naphthotriazoles

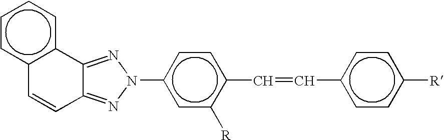

wherein R=—SO₃H, R′=H, and R=—CN and R′=—Cl;
the 2-(4-phenylstilben-4-yl)benzoxazoles 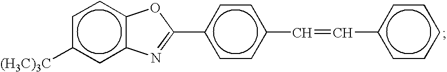
the bis(azol-2-yl)stilbenes 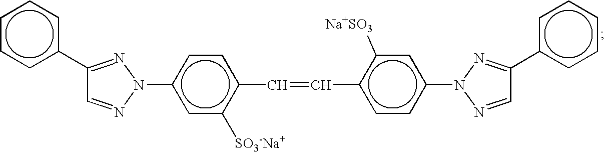
the 1,4-bis(styryl)benzenes 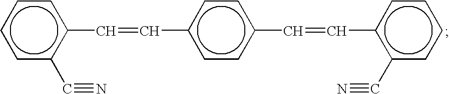
the 4,4′-bis(styryl)biphenyls 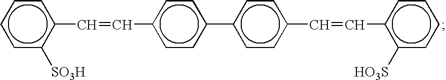
the 1,3-diphenyl-2-pyrazoline derivatives

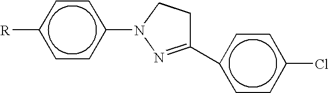

wherein R is —SO₃H, —SO₂NH₂, —SO₂NHCH₂CH₂₋CH₂N⁺(CH₃)₃⁻SO₃OCH₃, —SO₂CH₂CH₂SO₃H, sodium salt, or

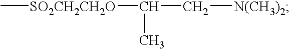

the bis(benzooxazol-2-yl) derivatives

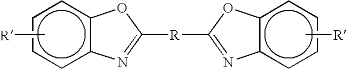

| R | R′ |
|---|---|
| —CH═CH— | alkyl, 5-CH₃ |
| 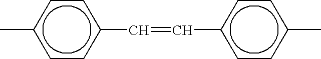 | H, alkyl |
| 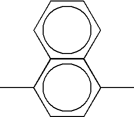 | H, alkyl |
| 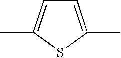 | COO-alkyl, SO₂-alkyl, H, alkyl |

the bis(benzimidazol-2-yl) derivatives 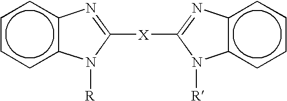
wherein X=—CH═CH— or 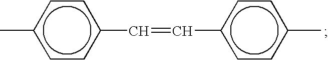
the 2-(benzofuran-2-yl)benzimidazoles;
the coumarins, including 3-phenyl-7-aminocoumarin, 3-phenyl-7-(azol-2-yl) coumarins, 3,7-bis(azolyl)-coumarins, 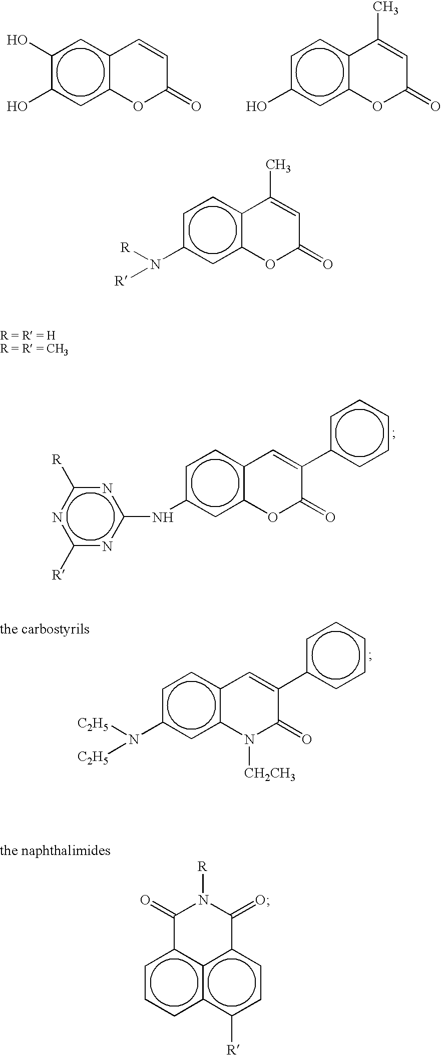
and miscellaneous compounds and classes such as quaternized pyridotriazoles, a pyrene compound 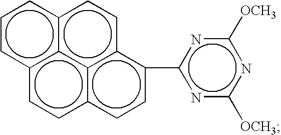
and the acylamino (R,R′) derivative of 3,7-diamino-dibenzothiophene-2,8-disulfonic acid-5,5-dioxide, wherein preferred acyl groups are alkoxybenzoyls,

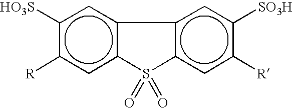

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

The topically active agent also can be a deodorant or antiperspirant compound, such as an astringent salt or a bioactive compound. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L_{z}, wherein z varies from about 0.9 to about 2 and is not necessarily an integer; n is the valence of L; 2−nz is greater than or equal to 0; and L is selected from the group consisting of halides, nitrate, sulfamate, sulfate, and mixtures thereof.

Exemplary deodorant compounds, therefore, include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, chlorophyllin copper complex, numerous other useful antiperspirant compounds listed in the CTFA Handbook at page 56, incorporated herein by reference, and mixtures thereof. The active agent also can be a fragrance that acts as a deodorizer by masking malodors. Numerous fragrance compounds are listed in the CTFA Handbook, pages 69-70, incorporated herein by reference.

In addition, other compounds can be included as the topically active agent in an amount sufficient to perform their intended function. For example, if the composition is intended to be a sunscreen, then compounds such as benzophenone-3, trihydroxycinnamic acid and salts, tannic acid, uric acids, quinine salts, dihydroxy naphtholic acid, an anthranilate, diethanolamine methoxycinnamate, p-aminobenzoic acid, phenylbenzimidazole sulfonic acid, PEG-25, p-aminobenzoic acid, or triethanolamine salicylate can be used as the active agent.

Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the active agent. Other sunscreen compounds are listed in CTFA Handbook, pages 86 and 87, incorporated herein by reference.

Similarly, topically active drugs, like antifungal compounds, antibacterial compounds, antiinflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidoneiodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin bleaching agents, skin protectants, such as allantoin, and antiacne agents, such as salicylic acid, also can be incorporated in a composition of the present invention in an amount sufficient to perform its intended function. Other topically active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

The active agent also can be a plant extract. Numerous plant extracts are available from Brucia Plant Extracts, Shingle Springs, Calif. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root.

Industrial products also can be used as the active agent. For example, catalysts can be incorporated into the expanded thermoplastic microspheres. Examples of catalysts include, but are not limited to, inorganic and organic catalysts, such as platinum, osmium, palladium, cobalt, copper, nickel, iron, zinc, tin, chloroplatinic acid, sulfuric acid, methanesulfonic acid, trifluoromethanesulfonic acid, and salts and complexes thereof.

Polymerization initiators and photoinitiators also can be used as the active agent. Photoinitiators are available commercially from Ciba Chemical Specialties under the tradenames DAROCURE® and IRGACURE®. Azopolymerization initiators are available from DuPont under the VAZO® tradename, e.g., VAZO® 52, VAZO® 64, VAZO® 67, and VAZO® 88. Peroxide polymerization initiators include ketone peroxides, peroxydicarbonates, peroxyesters, diacyl peroxide, dialkyl peroxides, hydroperoxides, and peroxyketals, all available commercially from Atofina Chemicals, Inc., Crosby, Tex., under the LUPEROX® tradename.

In the preparation of a sustained release composition of the present invention, the active agent is incorporated into the expanded thermoplastic microspheres. Additional active agents are disclosed in U.S. Pat. Nos. 5,145,675 and 5,851,538, each incorporated herein by reference.

3. Release Retardant

The control release capabilities of the sustained release composition may be improved by coating an optional release retardant on the expanded thermoplastic microspheres treated with the active agent. To help retard or eliminate premature displacement of the active agent from the expanded thermoplastic microspheres during storage or use, the optional release retardant typically is applied after the expanded thermoplastic microsphere-active agent combination is prepared. Alternatively, the optional release retardant can be added to the expanded thermoplastic microspheres simultaneously with the active agent.

The active agent-containing expanded thermoplastic microspheres also can be encapsulated by suspension or coacervation techniques known to persons skilled in the art using crosslinked polymers, like polyvinyl alcohol, acrylates, and methacrylates, or a colloid, like gelatin, agar, gum arabic, or carboxymethylcellulose.

In certain preferred embodiments, the release retardant is hydrophobic when the active agent is water soluble. Conversely, the release retardant preferably is hydrophilic when the active agent is oil soluble. These preferred combinations of active agent and release retardant are not essential to the present invention, because utilizing a hydrophilic release retardant with a water-soluble active agent, or a hydrophobic release retardant with an oil-soluble active agent, also improves the controlled release properties of the sustained release compositions.

The release retardant is adsorbed onto the expanded thermoplastic microspheres and also coats the expanded thermoplastic microspheres and the active agent. The release retardant, therefore, helps retard or eliminates a rapid displacement of the active agent from the expanded thermoplastic microspheres by water or a nonaqueous solvent, thereby leaving the active agent in an “incorporated” form that is available for controlled release.

The amount of release retardant present in a sustained release composition is 0% to about 80%, and preferably about 0.1% to about 60%, by weight of the composition. To achieve the full advantage of the present invention, the release retardant is present in an amount of 0% to about 50%, by weight of the composition.

The identity of the release retardant is not particularly limited. However, it is preferred that the release retardant is water insoluble, i.e., has a water-solubility of 0.1 g (gram) or less in 100 ml (milliliter) of water at 25° C., when the active agent is water soluble. It is also preferred that the release retardant is oil insoluble, i.e., has an oil-solubility of 0.1 g or less in 100 ml of mineral oil at 25° C., when the active agent is oil soluble. However, release retardants having oil or water solubility up to 20 g in 100 ml of mineral oil or water, respectively, can be used with either water-soluble and oil-soluble active agents.

The release retardant is selected such that it does not adversely affect the active agent, e.g., is nonreactive and noninteractive with the active agent. The release retardant can be a solid at room temperature, i.e., 25° C., or can be a liquid. A liquid release retardant has a low volatility, i.e., has a boiling point of above 150° C. at one atmosphere. In some embodiments, the release retardant can have cosmetic, medicinal, or other useful properties that perform in conjunction with the active agent.

Accordingly, one class of useful release retardants is the fatty alcohols, i.e., alcohols having eight through twenty carbon atoms (C₈-C₂₀). Fatty alcohols ethoxylated with one to three moles of ethylene oxide also are useful hydrophobic compounds. Examples of fatty alcohols and ethoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Sterols, like lanolin alcohol, also can be used as the release retardant. Additional fatty alcohols and sterols are listed in the CTFA Handbook, pages 28 and 45, incorporated herein by reference.

Another useful class of release retardants are the C₈-C₂₀ fatty acids, including, but not limited to, stearic acid, capric acid, behenic acid, caprylic acid, lauric acid, myristic acid, tallow acid, oleic acid, palmitic acid, isostearic acid, and additional fatty acids listed in the CTFA Handbook, pages 27 and 28, incorporated herein by reference.

Fats and oils also are useful release retardants. Examples of fats and oils include, but are not limited to, lanolin oil, linseed oil, coconut oil, olive oil, menhaden oil, castor oil, soybean oil, tall oil, rapeseed oil, palm oil, and neatsfoot oil. Glyceryl esters of fatty acids also can be used as the release retardant, as can lanolin derivatives, such as hydrogenated lanolin, oleyl lanolate, lanolinamide DEA, and similar lanolin derivatives. Similarly, essential oils, like eucalyptus oil, peppermint oil, rose oil, clove oil, lemon oil, pine oil, and orange oil, can be used as the release retardant. Such essential oils also can serve as a fragrance. Additional fats, oils, and essential oils are listed in the CTFA Handbook, pages 23, 26, and 27, incorporated herein by reference.

Other classes of useful release retardants include poly(acids), like poly(lactic acid); polymeric ethers, both homo and block copolymers, like poly(ethylene oxide-b-propylene oxide); polyols, like sorbitol, ascorbic acid, and mannitol; salts of C₈-C₂₀ fatty acids, e.g., sodium, potassium, aluminum, calcium, and magnesium salts of fatty acids; alkanolamides; and synthetic polymers, like a urea/formaldehyde resin, a polyethyleneimine, a polyacrylamide, a polyacrylic acid and salts thereof, polyvinylpyrrolidone and copolymers thereof, a polyisoprene, or a polystyrene, for example. Additional polymeric ethers, alkanolamides, and synthetic polymers are listed in the CTFA Handbook, pp. 3, 4, 38, 39, 47, and 48, incorporated herein by reference.

The release retardant also can be a biological polymer, a gum, a salt or derivative of a gum, or a carbohydrate. Examples of such release retardants include, but are not limited to, hyaluronic acid, potato starch, corn starch, rice starch, sodium hyaluronate, locust bean gum, tragacanth gum, xanthan gum, methylcellulose, hydroxyethylcellulose, karaya gum, carboxymethylcellulose, sucrose, sucrose laurate, dextrin, corn syrup, pectin, methyl gluceth-10, gelatin, algin, carrageenan, and mixtures thereof. Additional biological polymers, carbohydrates, gum, and salts and derivatives of gums are listed in the CTFA Handbook, at pp. 16, 19, 29, and 30, incorporated herein by reference.

Another class of release retardants is the sorbitan derivatives, like PEG-10 sorbitan laurate, PEG-20 sorbitan isostearate, PEG-3 sorbitan oleate, polysorbate 40, sorbitan stearate, and sorbitan palmitate, for example. Other sorbitan derivatives are listed in the CTFA Handbook, at page 44, incorporated herein by reference.

Another class of release retardants is the waxes, like mink wax, montan wax, carnauba wax, and candelilla wax, for example, and synthetic waxes, like silicone waxes, polyethylene, and polypropylene. Additional waxes are listed in the CTFA Handbook, pages 31 and 49, incorporated herein by reference.

4. Optional Ingredients

The sustained release compositions of the present invention are dry, powdery compositions. A present sustained release composition can be used as prepared, or can be incorporated into liquid, solid, or semisolid formulations. These formulations can be water based or oil based, and can be particulates, dispersions, emulsions, gels, or other physical forms known to persons skilled in the art.

These formulations can include optional ingredients traditionally included in cosmetic, medicinal, and other such compositions. These optional ingredients include, but are not limited to, dyes, fragrances, preservatives, antioxidants, detackifying agents, and similar types of compounds. The optional ingredients are included in the composition in an amount sufficient to perform their intended function.

Regardless of the particular expanded thermoplastic microspheres used, treatment of the microspheres with the active agent is readily accomplished by contact. The active agent can be dissolved in a solvent to form a solution which, in addition to facilitating incorporation, can be used to control the amount of active agents incorporated, control viscosity, and control any other parameters that can affect the quality and ease of incorporation. Examples of such solvents are liquid petrolatum, polysorbate ether, petroleum ether, alcohols (e.g., methanol, ethanol, propylene glycol, and higher alcohols), aromatics (e.g., benzene and toluene), alkanes (e.g., pentane, hexane, and heptane), ketones (e.g., acetone and methyl ethyl ketone), chlorinated hydrocarbons (e.g., chloroform, carbon tetrachloride, methylene chloride, and ethylene dichloride), and oils (e.g., isopropyl myristate, diisopropyl adipate, mineral oil, and silicone oils). After absorption of the solution, the solvent can be evaporated, or, if desired, can be retained with the active agent.

The active agent-containing expanded thermoplastic microspheres can be used as is, or can be incorporated into fluid or solid of the type commonly used for skin treatment, for example. These formulations include gels, creams, lotions, ointments, sprays, powders, oils, and sticks. Aqueous fluid compositions such as oil-in-water and water-in-oil emulsions, gels, creams, lotions, ointments, and sprays, where the sustained release compositions are dispersed in an aqueous medium, are preferred. Regardless of the formulation, however, the medium in which the sustained release compositions are dispersed can contain additional ingredients for any of a variety of cosmetic, therapeutic or preventive effects.

When sustained release compositions are suspended in aqueous or nonaqueous media, the concentration of the sustained release formulation relative to the entire formulation is sufficient for the active agent to perform its intended function, and typically is about 0.01% to about 50%, by weight, of the formulation.

The following examples illustrate sustained release compositions of the present invention.

### EXAMPLE 1

Adsorption capacity is the maximum weight percent of a liquid added to an adsorptive substrate (powder) until a very stiff, putty-like paste is produced. The adsorption capacity was determined by ASTM Method D 281-31, and the method disclosed in U.S. Pat. No. 4,962,170, incorporated herein by reference. In particular, the adsorption capacity is calculated from the weight difference of the powder containing the liquid and the dry powder according to the equation:

$$Adsorption Capacity ( \% ) = \frac{\begin{matrix}( wt . powder + liquid ) - \\ ( initial wt . powder ) \times 100\end{matrix}}{( wt . powder + liquid )}$$

In this test, EXPANCEL® 091 DE 40 d30 microspheres were tested for an ability to absorb water and mineral oil. It was found that the microspheres can absorb 20 grams of water per gram of particles (g/g) or 20 g/g of mineral oil.

### EXAMPLE 2

EXPANCEL® 091 DE 40 d30 microspheres of median particle size 20 microns were loaded with an isopropanol/lutein solution to a content of 2 grams per gram, and dried in a vacuum oven at 40° C. to evaporate the isopropanol. The dry sustained release composition was an orange, fine powder, containing 20% by weight entrapped lutein, i.e., 0.25 grams per gram of microspheres. The entrapped lutein was delivered as a free-flowing powder, and was stabilized against oxidation and degradation by light.

### EXAMPLE 3

A solution was prepared by dissolving 1 gram of urea peroxide in 1.5 grams of a water/acetone mixture. The solution was adsorbed on 1 gram of the EXPANCEL® 091 DE 40 d30 microspheres, then the water/acetone mixture was evacuated. The urea peroxide/microsphere sustained release composition was in form of a very fine, fluffy powder. Typically, urea peroxide is very unstable, shock sensitive, and deteriorates very quickly on contact with air humidity. The entrapped urea peroxide composition was stabilized and resisted degradation. The loading capacity of urea peroxide was 50 wt %, i.e., one gram per gram of microspheres.

### EXAMPLE 4

The EXPANCEL® 091 DE 40 d30 microparticles of median particle size 20 microns were loaded with a methanol/salicylic acid solution to a content 9.6 grams of solution per gram of microspheres. The resulting product was dried in an oven at 55° C. to evaporate the methanol. The dry, solid sustained release composition was a white, fine powder containing 60 wt % entrapped salicylic acid, i.e., 1.5 grams salicylic acid per gram of expanded thermoplastic microspheres.

### EXAMPLE 5

A solution was prepared by dissolving 1 gram of LEUCOPHOR® BSB (commercially available from Clariant Co.) fluorescent brightener in 19 grams of a 1:2 water/isopropyl alcohol solution. The resulting solution was adsorbed on 10 grams EXPANCEL® 091 DE 40 d30 microspheres of median particle size 20 microns. Then, the water/alcohol mixture was evaporated, and the resulting sustained release composition was pulverized. The sustained release composition can be used in cosmetic compositions to reduce the appearance of skin imperfections. Analysis showed that the expanded thermoplastic microspheres contained 0.2%, by weight, of the fluorescent brightener.

### EXAMPLE 6

VENUCEANE®, a natural antioxidant containing thermus ferment and glycerin, commercially available from Sederma (50 g), was entrapped in 50 g of EXPANCEL® 091 DE 40 d30 microspheres of median particle size 20 microns. A free-flowing powder was obtained. The sustained release composition can be used in cosmetic formulations for a sustained delivery of the natural antioxidant.

### EXAMPLE 7

The EXPANCEL® 091 DE 40 d30 microspheres were admixed with a zinc pyrithione slurry in water in an amount of 12 grams of the slurry per gram of the microspheres. The resulting sustained release formulation then was dried in an oven at 80° C. to evaporate water. The resulting dry product was a white, fine powder containing 85%, by weight, entrapped zinc pyrithione, i.e., 5.76 grams per gram of microspheres. Zinc pyrithione slurry as commercially available contains 51% water, 48% zinc pyrithione, and 1% of zinc chloride.

### EXAMPLE 8

Retinol was dissolved in hexane to a 50 wt % solution. The solution (10 grams) was adsorbed on 1 gram of the EXPANCEL® 091 DE 40 d30 microspheres. Then, the hexane was evaporated under vacuum, and a free-flowing powder was isolated. The adsorbed amount of retinol was about 70%, i.e., 2.4 grams per gram of the microspheres.

### EXAMPLE 9

EXPANCEL® 091 DE 40 d30 microspheres were loaded with a methanol/salicylic acid solution in an amount of 12 grams per gram of microspheres, and dried in an oven at 65° C. The resulting dry powder was a white fluffy powder containing 75% of entrapped salicylic acid, i.e., three grams of salicylic acid per gram of microspheres.

### EXAMPLE 10

Jojoba oil was freely incorporated into EXPANCEL® 091 DE 40 d30 microspheres by mixing in ratio 4:1, i.e., 80% of the active. A free-flowing white powder was obtained, which exhibited an excellent feel on skin and excellent dispersion and adsorption in the human skin, without any adverse side effects, like greasiness.

The sustained release compositions of the present invention are useful in the food, agricultural, personal care, cosmetic, manufacturing, and pharmaceutical industries. The present sustained release compositions provide a controlled release of topically active agents, flavorants, fragrances, pesticides, agricultural adjuvants, pigments, skin and hair color agents, catalysts, polymerization initiators, photoinitiators, and similar active agents.

Obviously, many modifications and variations of the invention as hereinbefore set forth can be made without departing from the spirit and scope thereof and, therefore, only such limitations should be imposed as are indicated by the appended claims.

1. A sustained release composition comprising:
(a) expanded thermoplastic microspheres;
(b) an active agent, said active agent incorporated into said expanded thermoplastic microspheres; and
(c) an optional release retardant, said release retardant coated over or adsorbed onto said expanded thermoplastic microspheres and said active agent.

2. The composition of claim 1 wherein the expanded thermoplastic microspheres are present in an amount of about 5% to about 99.9% by weight of the composition.

3. The composition of claim 1 wherein the expanded thermoplastic microspheres are present in an amount of about 10% to about 80% by weight of the composition.

4. The composition of claim 1 wherein the expanded thermoplastic microspheres have a median particle size of about 5 to about 250 microns.

5. The composition of claim 1 wherein the active agent is present in an amount of about 0.1% to about 95% by weight of the composition.

6. The composition of claim 1 wherein the active agent is water soluble.

7. The composition of claim 1 wherein the active agent is oil soluble.

8. The composition of claim 1 wherein the active agent is selected from the group consisting of a topically active compound, a fragrance, a plant extract, a spice, a food flavor, a food color, a catalyst, a polymerization initiator, a photoinitiator, a metal, a pigment, a pesticide, a drug, and a therapeutic agent.

9. The composition of claim 8 wherein the topically active agent is selected from the group consisting of a hair-growth promoter, a deodorant, an antiperspirant compound, a hair conditioner, a skin conditioner, an antioxidant, an insect repellant, a counterirritant, a vitamin, a plant extract, a steroid, a hair dye, a skin-lightening compound, a self-tanning compound, an antibacterial compound, an antifungal compound, an antiinflammatory compound, a topical anesthetic, an epidermal lipid replacement, a sunscreen, an optical brightener, a dermatitis or skin disease medication, and mixtures thereof.

10. The composition of claim 8 wherein the topically active agent is selected from the group consisting of benzocaine, dyclonine hydrochloride, aloe vera, butamben picrate, lidocaine hydrochloride, xylocaine, providone-iodine, polymyxin b sulfate-bacitracin, zinc-neomycin sulfate-hydrocortisone, chloramphenicol, ethylbenzethonium chloride, erythromycin, lindane, benzoyl peroxide, erythromycin benzoyl peroxide, clindamycin phosphate, 5,7-dichloro-8-hydroxyquinoline, alclometasone dipropionate, betamethasone valerate, o-amino-p-toluenesulfonamide monoacetate, monobenzone, amcinonide, diflorasone diacetate, hydrocortisone, methylbenzethonium chloride, PEG-4 dilaurate, lanolin oil, petrolatum, mineral wax, butocouazole nitrate, haloprogin, clotrimazole, O-[(2-hydroxymethyl)methyl]guanine, alclometasone dipropionate, betamethasone valerate, isopropyl myristate MSD, anthralin, methoxsalen, coal tar, 2-(acetyloxy)-9-fluoro-1′,2′,3′,4′-tetrahydro-11-hydroxypregna-1,4-dieno-[16,17-b]naphthalene-3,20-dione, 21-chloro-9-fluoro-1′,2′,3′,4′-tetrahydro-11b-hydroxypregna-1,4-dieno-[16z,17-b]naphthalene-3,20-dione, allantoin, salicylic acid, and mixtures thereof.

11. The composition of claim 1 wherein the active agent is selected from the group consisting of marigold extract, urea peroxide, thermus ferment, glycerin, an optical brightener, an antioxidant, a silicone, isopropyl myristate, vitamin E acetate, ascorbic acid, retinol, salicylic acid, zinc pyrithione, benzophenone-3, a fragrance, glycolic acid, hyalauronic acid, hydrogen peroxide, a protein, an enzyme, tocopherol, lutein, hydroquinone, kojic acid, jojoba oil, an alpha or beta hydroxy acid, and mixtures thereof.

12. The composition of claim 1 wherein the release retardant is present in an amount of about 0.1% to about 80% by weight of the composition.

13. The composition of claim 12 wherein the release retardant is water insoluble, and is selected from the group consisting of a fatty alcohol, a C₈-C₂₀ fatty acid, a fat, an oil, and mixtures thereof.

14. The composition of claim 11 wherein the release retardant is water soluble, and is selected from the group consisting of a poly(acid), a polyol, a salt of a C₈-C₂₀ fatty acid, an alkanolamide, a water-soluble polymer, a biological polymer, a gum, a carbohydrate, a cellulose derivative, a sorbitan derivative, and mixtures thereof.

15. A formulation comprising a sustained release composition of claim 1 and a carrier.

16. The formulation of claim 15 wherein the formulation is a liquid.

17. The formulation of claim 15 wherein the formulation is a solid.

18. The formulation of claim 15 wherein the formulation is a semisolid.

19. A method of treating skin or hair comprising a step of contacting the skin or hair with a sustained release composition comprising:
(a) expanded thermoplastic microspheres;
(b) a topically active agent, said topically active agent incorporated into said expanded thermoplastic microspheres; and
(c) an optional release retardant, said release retardant coated over or adsorbed onto said expanded thermoplastic microspheres and said topically active agent.
